SEQUENCE LISTING (5 sequences)

SEQ ID NO: 1 - protein, 21 aa, Artificial Sequence
  source: /note=“Description of Artificial Sequence Synthetic peptide”
  SITE (1)..(3): /note=“This region may or may not be present”
GSGEGRGSLLTCGDVEENPGP

SEQ ID NO: 2 - protein, 22 aa, Artificial Sequence
  source: /note=“Description of Artificial Sequence Synthetic peptide”
  SITE (1)..(3): /note=“This region may or may not be present”
GSGATNFSLLKQAGDVEENPGP

SEQ ID NO: 3 - protein, 23 aa, Artificial Sequence
  source: /note=“Description of Artificial Sequence Synthetic peptide”
  SITE (1)..(3): /note=“This region may or may not be present”
GSGQCTNYALLKLAGDVESNPGP

SEQ ID NO: 4 - protein, 25 aa, Artificial Sequence
  source: /note=“Description of Artificial Sequence Synthetic peptide”
  SITE (1)..(3): /note=“This region may or may not be present”
GSGVKQTLNFDLLKLAGDVESNPGP

SEQ ID NO: 5 - protein, 9 aa, Unknown
  source: /note=“Description of Unknown 2A conserved sequence”
  MOD_RES (5)..(5): Any amino acid
GDVEXNPGP

(19) United States
(12) Patent Application Publication
(10) Pub. No.: US 2022/0033848 A1
(43) Pub. Date: Feb. 3, 2022

(54) A MODULAR, POLYCISTRONIC VECTOR FOR CAR AND TCR TRANSDUCTION

(71) Applicant: Board of Regents, The University of Texas System, Austin, TX (US)

(72) Inventors: Katy Rezvani, Houston, TX (US); Elizabeth Shpall, Houston, TX (US)

(21) Appl. No.: 17/309,316

(22) PCT Filed: Nov. 18, 2019
(86) PCT No.: PCT/US19/62014

§ 371 Date: May 18, 2021

Related U.S. Application Data
(60) Provisional application No. 62/769,414, filed on Nov. 19, 2018.
(60) Provisional application No. 62/773,394, filed on Nov. 30, 2018.
(60) Provisional application No. 62/791,491, filed on Jan. 11, 2019.

(51) Int. Cl. C12N 15/86; A61K 35/17

(52) U.S. Cl. CPC C12N 15/86; A61K 35/17

(57) ABSTRACT

Provided herein is a modular polycistronic vector system, such as for the genetic reprogramming of cells to express one or more antigen receptors. The modular characteristic of the system allows for exchange of one or more cistrons in addition to one or more components within particular cistrons. In specific embodiments, the modularity of the system allows exchange of components of a chimeric antigen receptor, such as exchange of costimulatory domains, scFvs, hinges, signaling domains, and so forth.

This application claims priority to U.S. Provisional Patent Application Ser. No. 62/769,414, filed Nov. 19, 2018; U.S. Provisional Patent Application Ser. No. 62/773,394, filed Nov. 30, 2018; and U.S. Provisional Patent Application Ser. No. 62/791,491, filed Jan. 11, 2019, all of which applications are incorporated by reference herein in their entirety.

### SEQUENCE LISTING

The instant application contains a Sequence Listing which has been submitted electronically in ASCII format and is hereby incorporated by reference in its entirety. Said ASCII copy, created on Nov. 13, 2019, is named UTFC_P1152WO_SL.txt and is 2,502 bytes in size.

### BACKGROUND

### 1. Field

The present disclosure relates generally at least to the fields of immunology, cell biology, molecular biology, recombinant technology, and medicine. More particularly, it concerns a polycistronic vector, such as for the expression of antigen receptor(s).

### 2. Description of Related Art

Despite remarkable technological advancements in the diagnosis and treatment options available to patients diagnosed with cancer, the prognosis still often remains poor and many patients cannot be cured. Immunotherapy holds the promise of offering a potent, yet targeted, treatment to patients diagnosed with various tumors with the potential to eradicate the malignant tumor cells without damaging normal tissues. In theory, the T cells of the immune system are capable of recognizing protein patterns specific for tumor cells and to mediate their destruction through a variety of effector mechanisms.

Genetic reprogramming of immune cells, such as Natural Killer (NK) cells, for adoptive cancer immunotherapy has clinically relevant applications and benefits such as innate anti-tumor surveillance without prior need for sensitization allogeneic efficacy without graft versus host reactivity, and direct cell-mediated cytotoxicity and cytolysis of target tumors. The administration of immune cells expressing chimeric antigen receptors (CARs), such as adoptive T cell therapy or NK cells therapy, is an attempt to harness and amplify the tumor-eradicating capacity of a patient's own immune cells and then return these effectors to the patient in such a state that they effectively eliminate residual tumor, however without damaging healthy tissue. Commonly, CARs comprise a single chain variable fragment (scFv) of an antibody specific for a tumor associated antigen (TAA) coupled via hinge and transmembrane regions to cytoplasmic domains of T-cell signaling molecules.

Co-expression of multiple genes at a desired ratio is highly attractive for a broad array of basic research and biomedical applications including genetic reprogramming. Strategies for multigene co-expression include introduction of multiple vectors, use of multiple promoters in a single vector, fusion proteins, proteolytic cleavage sites between genes, internal ribosome entry sites, and “self-cleaving” 2A peptides. However, there is an unmet need for methods for the combinatorial expression of multiple genes including CARs, T cell receptor (TCRs), cytokines, cytokine receptors, chemokine receptors, and homing receptors, to name a few. The present disclosure satisfies that need.

### SUMMARY

Embodiments of the disclosure include methods and compositions related to polycistronic vectors comprising at least two, at least three, or at least four cistrons each flanked by one or more restriction enzyme sites, wherein at least one cistron encodes for at least one antigen receptor. In some cases, two, three, four, or more of the cistrons are translated into a single polypeptide and cleaved into separate polypeptides. Adjacent cistrons on the vector may be separated by a site that provides for the ability of encoded polypeptides to be separate molecules. For example, adjacent cistrons on the vector may be separated by a self cleavage site, such as a 2A self cleavage site. In some cases, each of the cistrons express separate polypeptides from the vector. On particular cases, adjacent cistrons on the vector are separated by an IRES element.

In particular embodiments of the disclosure, at least one of the cistrons on the vector comprises two or more modular components, wherein each of the modular components within a cistron is flanked by one or more restriction enzyme sites. A cistron may comprise three, four, or five modular components, for example. In at least some cases, a cistron encodes an antigen receptor having different parts of the receptor encoded by corresponding modular components. A first modular component of a cistron may encode an antigen binding domain of the receptor. In addition, a second modular component of a cistron may encode a hinge region of the receptor. In addition, a third modular component of a cistron may encode a transmembrane domain of the receptor. In addition, a fourth modular component of a cistron may encode a first costimulatory domain. In addition, a fifth modular component of a cistron may encode a second costimulatory domain. In addition, a sixth modular component of a cistron may encode a signaling domain.

In particular aspects of the disclosure, two different cistrons on the vector each encode non-identical antigen receptors. Both antigen receptors may be encoded by a cistron comprising two or more modular components, including separate cistrons comprising two or more modular components. The antigen receptor may be a chimeric antigen receptor (CAR) and/or T cell receptor (TCR), for example.

In specific embodiments, the vector is a viral vector (retroviral vector, lentiviral vector, adenoviral vector, or adeno-associated viral vector, for example) or a non-viral vector. The vector may comprise a Moloney Murine Leukemia Virus (MMLV) 5′ LTR, 3′ LTR, and/or psi packaging element. In specific cases, the psi packaging is incorporated between the 5′ LTR and the antigen receptor coding sequence. The vector may or may not comprise pUC19 sequence. In some aspects of the vector, at least one cistron encodes for a secreted or membrane-bound cytokine (interleukin 15 (IL-15), IL-7, IL-21, IL-12, IL-18, or IL-2, for example), chemokine, cytokine receptor, and/or homing receptor.

When 2A cleavages sites are utilized in the vector, the 2A cleavage site may comprise a P2A, T2A, E2A and/or F2A site.

Any cistron of the vector may comprise a suicide gene. Any cistron of the vector may encode a reporter gene. In specific embodiments, a first cistron encodes a suicide gene, a second cistron encodes an antigen receptor, a third cistron encodes a reporter gene, and a fourth cistron encodes a cytokine. In certain embodiments, a first cistron encodes a suicide gene, a second cistron encodes a first antigen receptor, a third cistron encodes a second antigen receptor, and a fourth cistron encodes a cytokine. In specific embodiments, different parts of the antigen receptor are encoded by corresponding modular components and a first component of the second cistron encodes an antigen binding domain, a second component encodes a hinge and/or transmembrane domain, a third component encodes a costimulatory domain, and a fourth component encodes a signaling domain.

The methods and compositions of the disclosure encompass any suitable order of cistrons on a single vector.

Embodiments of the disclosure include any kind of cells that harbor any of the vectors encompassed herein. In specific embodiments, there is an immune cell that comprises a vector of the disclosure, and the immune cell may be a T cell, peripheral blood lymphocyte, B cell, NK cell, invariant NK cell, NKT cell, iNKT, macrophage, or stem cell (such as a mesenchymal stem cell (MSC) or an induced pluripotent stem (iPS) cell). The T cell may be a CD8+ T cell, CD4+ T cell, or gamma-delta T cell. In specific embodiments, the T cell is a cytotoxic T lymphocyte (CTL). Any cells may be allogeneic or autologous with respect to an individual. The immune cells may be human cells. The immune cell may be derived from cord blood, peripheral blood, bone marrow, CD34+ cells, or iPSCs. A plurality of immune cells may be comprised as a population of cells. In specific cases, the immune cells are comprised in a pharmaceutically acceptable carrier.

In one embodiment of the disclosure, there is a method for producing immune cells, comprising (a) obtaining a starting population of immune cells; (b) culturing the starting population of immune cells in the presence of artificial presenting cells (APCs); (c) introducing a vector encompassed in the disclosure into the immune cells; and (d) expanding the immune cells in the presence of APCs, thereby obtaining expanded immune cells. The starting population of immune cells may be obtained by isolating mononuclear cells using a ficoll-paque density gradient. The APCs may be gamma-irradiated APCs. In some cases of the method, the method further comprises cryopreserving a population of the expanded immune cells.

Embodiments of the disclosure include a pharmaceutical composition comprising any population of immune cells as encompassed herein and a pharmaceutically acceptable carrier.

In particular embodiments, there is provided a composition comprising an effective amount of immune cells as encompassed herein for use in the treatment of a disease or disorder in an individual, such as cancer or an immune-related disorder. In specific embodiments, there is use of a composition comprising an effective amount of immune cells encompassed by the disclosure for the treatment of cancer or an immune-related disorder in an individual.

In one embodiment, there is a method of treating a disease or disorder in an individual, comprising administering an effective amount of immune cells encompassed by the disclosure to the individual. In specific embodiments, the disease or disorder is a cancer (solid cancer or hematologic malignancy), autoimmune disorder, graft versus host disease, allograft rejection, or inflammatory condition. The autoimmune disorder may be an inflammatory condition and the immune cells may have essentially no expression of glucocorticoid receptor. The subject has been or may be being administered a steroid therapy. The immune cells may be autologous or allogeneic with respect to the individual. The method may further comprise administering at least a second therapeutic agent to the individual, such as chemotherapy, immunotherapy, surgery, radiotherapy, or biotherapy. In specific embodiments, the immune cells are administered to the individual intravenously, intraperitoneally, intratracheally, intratumorally, intramuscularly, endoscopically, intralesionally, percutaneously, subcutaneously, regionally, by perfusion, or by direct injection. In particular embodiments, the second therapeutic agent is administered to the individual intravenously, intraperitoneally, intratracheally, intratumorally, intramuscularly, endoscopically, intralesionally, percutaneously, subcutaneously, regionally, by perfusion, or by direct injection.

In some embodiments, polycistronic vectors comprise at least two, at least three, or at least four cistrons each flanked by one or more restriction enzyme sites, wherein at least one of the cistrons on the vector comprises two or more modular components, wherein each of the modular components within a cistron is flanked by one or more restriction enzyme sites.

Other objects, features and advantages of the present disclosure will become apparent from the following detailed description. It should be understood, however, that the detailed description and the specific examples, while indicating particular embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A: One embodiment of a polycistronic vector comprising multiple modular cistrons with at least one cistron having multiple modular components. X represents at least one restriction enzyme site.

FIG. 1B: Another embodiment of a polycistronic vector comprising multiple modular cistrons with at least one cistron having multiple modular components. X represents at least one restriction enzyme site.

FIG. 2: Schematic depicting a linear representation of one example of a polycistronic retroviral vector with four cistrons expressing 7 open reading frames (ORFs) (including components within an antigen receptor cistron) via three 2A self-cleavage sites.

FIG. 3: One example of a polycistronic plasmid with multiple cistrons and three 2A cleavage sites.

### DESCRIPTION OF ILLUSTRATIVE EMBODIMENTS

In certain embodiments, the present disclosure provides a flexible, modular system utilizing a polycistronic vector having the ability to express multiple cistrons at substantially identical levels. The system may be used for cell engineering allowing for combinatorial expression (including overexpression) of multiple genes. In specific embodiments, one or more of the genes expressed by the vector includes one, two, or more antigen receptors that in specific embodiments are non-natural receptors. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise: (1) one or more reporters, for example fluorescent or enzymatic reporters, such as for cellular assays and animal imaging; (2) one or more cytokines or other signaling molecules; and/or (3) a suicide gene.

Specifically, the vector may comprise at least 4 cistrons separated by cleavage sites of any kind, such as 2A cleavage sites. The vector may or may not be Moloney Murine Leukemia Virus (MoMLV or MMLV)-based including the 3′ and 5′ LTR with the psi packaging sequence in a pUC19 backbone. The vector may comprise 4 or more cistrons with three or more 2A cleavage sites and multiple ORFs for gene swapping. The system allows for combinatorial overexpression of multiple genes (7 or more) that are flanked by restriction site(s) for rapid integration through subcloning, and the system also includes at least three 2A self-cleavage sites, in some embodiments. Thus, the system allows for expression of multiple CARs, TCRs, signaling molecules, cytokines, cytokine receptors, and/or homing receptors. This system may also be applied to other viral and non-viral vectors, including but not limited lentivirus, adenovirus AAV, as well as non-viral plasmids.

The modular nature of the system also enables efficient subcloning of a gene into each of the 4 cistrons in the polycistronic expression vector and the swapping of genes, such as for rapid testing. Restriction sites strategically located in the polycistronic expression vector allow for swapping of genes with efficiency. Examples of restriction enzyme sites include at least the following: Bln I, BstE II, EcoR V, Acc I, Alu I, Apa I, BamH I, Bcl I, Bgl II, Bsm I, Cfo I, Cla I, Dde I, Dpn I, Dra I, EclX I, EcoR I, Hae III, Hind III, Hpa I, Kpn I, Ksp I, Mva I, Nco I, Nde I, Nhe I, Not I, Nsi I, Pst I, Pvu I, Pvu II, Rsa I, Sac I, Sal I, Sau3A I, Sca I, Sfi I, Sma I, Spe I, Sph I, Taq I, Xba I, and/or Xho I.

Further provided herein are methods for genetically engineering immune cells with the modular vectors encompassed herein. The immune cells may be of any kind, including T cells, B cells, NK cells, NKT cells, or mesenchymal stromal cells (MSCs), for example. Also provided herein are methods of immunotherapy comprising administration of the vector-engineered immune cells, such as for the treatment of cancer, infection, or autoimmune diseases, for example.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein embodiments which are disclosed and still obtain a like or similar result without departing from the spirit and scope of the disclosure. The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

The term “cistron” as used herein refers to a nucleic acid sequence from which a gene product may be produced.

The term “modular” as used herein refers to a cistron or component of a cistron that allows for interchangeability thereof, such as by removal and replacement of an entire cistron or of a component of a cistron, respectively, for example by using standard recombination techniques, including utilizing one or more restriction enzyme sites, including unique restriction enzyme sites in some cases.

Throughout this application, the term “about” is used to indicate that a value includes the inherent variation of error for the device, the method being employed to determine the value, or the variation that exists among the study subjects.

An “immune disorder,” “immune-related disorder,” or “immune-mediated disorder” refers to a disorder in which the immune response plays a key role in the development and/or progression of the disease. Immune-mediated disorders include autoimmune disorders, allograft rejection, graft versus host disease and inflammatory and allergic conditions, for example.

An “immune response” is a response of a cell of the immune system, such as a B cell, or a T cell, or innate immune cell to a stimulus. In one embodiment, the response is specific for a particular antigen (an “antigen-specific response”).

An “autoimmune disease” refers to a disease in which the immune system produces an immune response (for example, a B-cell or a T-cell response) against an antigen that is part of the normal host (that is, an autoantigen), with consequent injury to tissues. An autoantigen may be derived from a host cell or may be derived from a commensal organism, such as the micro-organisms (known as commensal organisms) that normally colonize mucosal surfaces.

“Treating” or treatment of a disease or condition refers to executing a protocol, which may include administering one or more drugs or therapies (including cells) to a patient, in an effort to alleviate at least one sign or symptom of the disease. Desirable effects of treatment include decreasing the rate of disease progression, ameliorating or palliating the disease state, delaying the onset of at least one symptom, and remission or improved prognosis. Alleviation can occur prior to signs or symptoms of the disease or condition appearing, as well as after their appearance, or both. Thus, “treating” or “treatment” may include “preventing” or “prevention” of disease or undesirable condition. In addition, “treating” or “treatment” does not require complete alleviation of one or more signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of the condition. This includes, but is not limited to, a reduction in the frequency or severity of one or more signs or symptoms of a disease. For example, treatment of cancer may involve, for example, a reduction in the size of a tumor, a reduction in the invasiveness of a tumor, reduction in the growth rate of the cancer, and/or prevention of metastasis. Treatment of cancer may also refer to prolonging survival of a subject with cancer.

“Subject” and “patient” and “individual” refer to either a human or non-human, such as primates, mammals, and vertebrates. In particular embodiments, the subject is a human, dog, cat, horse, cow, and so forth.

The phrases “pharmaceutically acceptable or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “antigen presenting cells (APCs)” refers to a class of cells capable of presenting one or more antigens in the form of a peptide-MHC complex recognizable by specific effector cells of the immune system, and thereby inducing an effective cellular immune response against the antigen or antigens being presented. The term “APC” encompasses intact whole cells such as macrophages, B-cells, endothelial cells, activated T-cells, and dendritic cells, or molecules, naturally occurring or synthetic capable of presenting antigen, such as purified MHC Class I molecules complexed to beta2-microglobulin.

### II. POLYCISTRONIC MODULAR VECTOR SYSTEM

Embodiments of the disclosure encompass systems that utilize a polycistronic vector wherein at least part of the vector is modular, for example by allowing removal and replacement of one or more cistrons (or component(s) of one or more cistrons), such as by utilizing one or more restriction enzyme sites whose identity and location are specifically selected to facilitate the modular use of the vector. The vector also has embodiments wherein multiple of the cistrons are translated into a single polypeptide and processed into separate polypeptides, thereby imparting an advantage for the vector to express separate gene products in substantially equimolar concentrations.

The vector of the disclosure is configured for modularity to be able to change one or more cistrons of the vector and/or to change one or more components of one or more particular cistrons. The vector may be designed to utilize unique restriction enzyme sites flanking the ends of one or more cistrons and/or flanking the ends of one or more components of a particular cistron.

In certain embodiments, the present disclosure provides a system for cell engineering allowing for combinatorial expression, including overexpression, of multiple cistrons that may include one, two, or more antigen receptors, for example. In particular embodiments, the use of a polycistronic vector as described herein allows for the vector to produce equimolar levels of multiple gene products from the same mRNA. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise one or more fluorescent or enzymatic reporters, such as for cellular assays and animal imaging. The vector may also comprise a suicide gene product for termination of cells harboring the vector when they are no longer needed or become deleterious to a host to which they have been provided.

In specific cases, the vector may be a γ-retroviral transfer vector. The retroviral transfer vector may comprises a backbone based on a plasmid, such as the pUC19 plasmid (large fragment (2.63 kb) in between HindIII and EcoRI restriction enzyme sites). The backbone may carry viral components from Moloney Murine Leukemia Virus (MoMLV) including 5′ LTR, psi packaging sequence, and 3′ LTR. LTRs are long terminal repeats found on either side of a retroviral provirus, and in the case of a transfer vector, brackets the genetic cargo of interest, such as CARs and associated components. The psi packaging sequence, which is a target site for packaging by nucleocapsid, is also incorporated in cis, sandwiched between the 5′ LTR and the CAR coding sequence. Thus, the basic structure of an example of a transfer vector can be configured as such: pUC19 sequence-5′ LTR-psi packaging sequence-genetic cargo of interest-3′ LTR-pUC19 sequence. This system may also be applied to other viral and non-viral vectors, including but not limited lentivirus, adenovirus AAV, as well as non-viral plasmids.

In particular embodiments, multiple cistrons of the vector are separated by one or more elements that provide for expression of genes from the corresponding multiple cistrons into a single transcript. The single transcript is subsequently translated to produce a multi-protein polypeptide that is processed (for example, by cleavage) such that the proteins become separate protein molecules. An exemplary element is a site that encodes a self-cleaving peptide, such as a 2A peptide cleavage sequence. Other cleavage sites include furin cleavage site or a Tobacco Etch Virus (TEV) cleavage site. In other cases, the cistrons of the vector are separated by one or more elements that provide for distinct translation of the separate cistrons (such as IRES sequences). In some cases, the vector utilizes a combination of both types of elements.

The genetic cargo of interest may include 1, 2, 3, 4, 5, 6, 7, 8, 9, 10, or more cistrons comprising at least one ORF that may be expressed from the vector. Embodiments of the disclosure include the vector in states wherein the genetic cargo of interest may not be presently housed in the vector but the vector still retains one or more structural or housekeeping elements required for expression and/or further processing of cistrons when they are present (such as promoter(s), multiple 2A sequences, etc.). The vector may have multiple cistrons that are able to be translated into a single polypeptide and processed into separate polypeptides (such as by using 2A self cleavage sites between adjacent cistrons). In alternative embodiments, multiple of the cistrons are expressed as separate polypeptides (such as by using IRES elements between adjacent cistrons).

In specific cases, the structure of the genetic cargo of interest in the vector may be as follows:

Cistron 1-2A-Cistron 2-2A-Cistron 3-2A-Cistron 4,

wherein in specific embodiments the cistron 1, cistron 2, cistron 3, and cistron 4 are different genes. In at least some cases, the 2A sequences within a vector may or may not be identical.

In specific embodiments, at least one of the cistrons encodes a suicide gene. In some embodiments, at least one of the cistrons encodes a cytokine. In certain embodiments, at least one cistron encodes an antigen receptor. A cistron may or may not encode a reporter gene. In certain embodiments, at least two cistrons encode two different antigen receptors (e.g., CARs and/or TCRs). A cistron may or may not encode a reporter gene. In particular cases, the genetic cargo of interest is as follows:
suicide gene-2A-antigen receptor-2A-reporter gene-2A-cytokine; or
suicide gene-2A-antigen receptor-2A-cytokine-2A reporter gene; or
suicide gene-2A-cytokine-2A reporter gene-2A-antigen receptor; or
suicide gene-2A-cytokine-2A antigen receptor-2A-reporter gene; or
suicide gene-2A-reporter gene-2A-cytokine-2A antigen receptor; or
suicide gene-2A-reporter gene-2A-antigen receptor-2A-cytokine; or
antigen receptor-2A-cytokine-2A-reporter gene-2A-suicide gene; or
antigen receptor-2A-cytokine-2A-suicide gene-2A-reporter gene; or
antigen receptor-2A-reporter gene-2A-cytokine-2A-suicide gene; or
antigen receptor-2A-reporter gene-2A-suicide gene-2A-cytokine; or
antigen receptor-2A-suicide gene-2A-reporter gene-2A-cytokine; or
antigen receptor-2A-suicide gene-2A-cytokine-2A-reporter gene; or
reporter gene-2A-antigen receptor-2A-suicide gene-2A-cytokine; or
reporter gene-2A-antigen receptor-2A-cytokine-2A-suicide gene; or
reporter gene-2A-cytokine-2A-suicide gene-2A-antigen receptor; or
reporter gene-2A-cytokine-2A-antigen receptor-2A-suicide gene; or
reporter gene-2A-suicide gene-2A-antigen receptor-2A-cytokine; or
reporter gene-2A-suicide gene-2A-cytokine-2A-antigen receptor; or
cytokine-2A-reporter gene-2A-suicide gene-2A-antigen receptor; or
cytokine-2A-reporter gene-2A-antigen receptor-2A-suicide gene; or
cytokine-2A-suicide gene-2A-antigen receptor-2A-reporter gene; or
cytokine-2A-suicide gene-2A-reporter gene-2A-antigen receptor; or
cytokine-2A-antigen receptor-2A-suicide gene-2A-suicide gene; or
cytokine-2A-antigen receptor-2A-reporter gene-2A-reporter gene.

In particular configurations of the genetic cargo of interest, a single vector may comprise a cistron that encodes a first antigen receptor and a cistron that encodes a second antigen receptor that is non-identical to the first antigen receptor. In specific embodiments, the first antigen receptor encodes a CAR and the second antigen receptor encodes a TCR, or vice versa. In particular embodiments, a vector comprising separate cistrons that respectively encode a first antigen receptor and a second antigen receptor also comprises a third cistron that encodes a cytokine or chemokine and a fourth cistron that encodes a suicide gene. However, the suicide gene and/or the cytokine (or chemokine) may not be present on the vector.

In particular cases, the genetic cargo of interest is as follows:
suicide gene-2A-first antigen receptor-2A-cytokine-2A-second antigen receptor; or
suicide gene-2A-first antigen receptor-2A-second antigen receptor-2A-cytokine; or
suicide gene-2A-cytokine-2A-first antigen receptor-2A-second antigen receptor; or
suicide gene-2A-cytokine-2A-second antigen receptor-2A-first antigen receptor; or
suicide gene-2A-second antigen receptor-2A-first antigen receptor-2A-cytokine; or
suicide gene-2A-second antigen receptor-2A-cytokine-2A-first antigen receptor; or
first antigen receptor-2A-cytokine-2A-second antigen receptor-2A-suicide gene; or
first antigen receptor-2A-cytokine-2A-suicide gene-2A-second antigen receptor; or
first antigen receptor-2A-suicide gene-2A-second antigen receptor-2A-cytokine; or
first antigen receptor-2A-suicide gene-2A-cytokine-2A-second antigen receptor; or
first antigen receptor-2A-second antigen receptor-2A-cytokine-2A-suicide gene; or
first antigen receptor-2A-second antigen receptor-2A-suicide gene-2A-cytokine; or
cytokine-2A-first antigen receptor-2A-suicide gene-2A-second antigen receptor; or
cytokine-2A-first antigen receptor-2A-second antigen receptor-2A-suicide gene; or
cytokine-2A-suicide gene-2A-first antigen receptor-2A-second antigen receptor; or
cytokine-2A-suicide gene-2A-second antigen receptor-2A-first antigen receptor; or
cytokine-2A-second antigen receptor-2A-first antigen receptor-2A-suicide gene; or
cytokine-2A-second antigen receptor-2A-suicide gene-2A-first antigen receptor.

In some cases, one of the cistrons in the vector is not a reporter gene or is not a cytokine. In alternative embodiments to the configuration of the cistrons provided above, instead of a 2A peptide sequence there may be an alternative element to allow for co-expression of different cistrons, such as an IRES sequence. In a single vector, there may be a combination of 2A peptide cleavage sequences and IRES elements utilized.

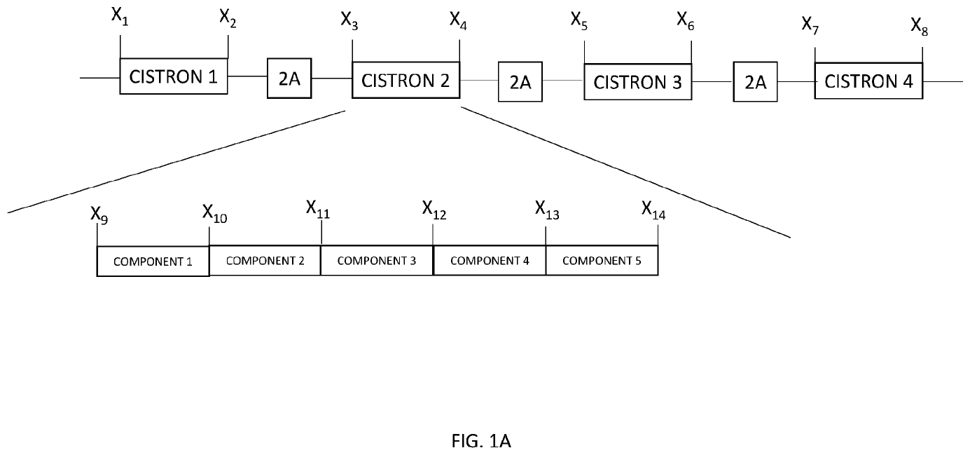

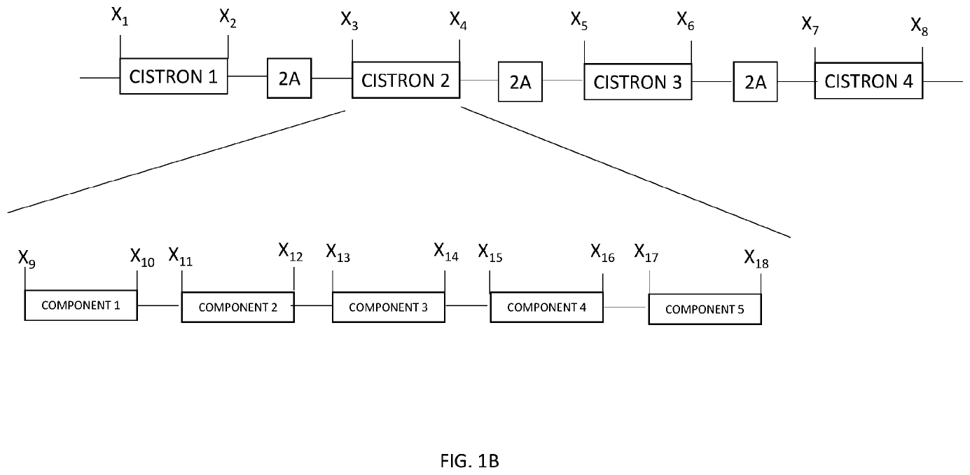

In particular embodiments, at least one cistron comprises multiple component(s) themselves that are modular. For example, one cistron may encode a multi-component gene product, such as an antigen receptor having multiple parts; in specific cases the antigen receptor is encoded from a single cistron, thereby ultimately producing a single polypeptide. The cistron encoding multiple components may have the multiple components separated by 1, 2, 3, 4, 5, or more restriction enzyme digestion sites, including 1, 2, 3, 4, 5, or more restriction enzyme digestion sites that are unique to the vector comprising the cistron (FIGS. 1A and 1B). In specific embodiments, a cistron having multiple components encodes an antigen receptor having multiple corresponding parts each attributing a unique function to the receptor. In a specific embodiment, each or the majority of components of the multi-component cistrons is separated by one or more restriction enzyme digestion sites that are unique to the vector, allowing the interchangeability of separate components when desired.

As an illustration, the modularity of one example of a multi-component cistronis configured as follows, wherein there are one or more unique restriction enzyme sites as represented by each X:

component 1-X₁-component 2-X₂-component 3-X₃-component 4-X₄-component 5-X₅- etc.

In specific embodiments, each component of a multi-component cistron corresponds to a different part of an encoded antigen receptor, such as a chimeric antigen receptor (CAR). In illustrative embodiments, component 1 may encode an antigen binding domain of the receptor; component 2 may encode a hinge domain of the receptor; component 3 may encode a transmembrane domain of the receptor; component 4 may encode a costimulatory domain of the receptor, and component 5 may encode a signaling domain of the receptor. In specific embodiments, an antigen receptor may comprise one or more costimulatory domains, each separated by unique restriction enzyme digestion sites for interchangeability of the costimulatory domain(s) within the receptor.

Turning to FIGS. 1A and 1B, these illustrations provide examples of embodiments of at least part of one vector of the disclosure and show the modular nature of the vector. FIG. 1A illustrates a polycistronic vector having four separate cistrons where adjacent cistrons are separated by a 2A cleavage site, although in specific embodiments instead of a 2A cleavage site there is an element that directly or indirectly causes separate polypeptides to be produced from the cistrons (such as an IRES sequence). In FIG. 1A, four separate cistrons are separated by three 2A peptide cleavage sites, and each cistron has restriction sites (X₁, X₂, etc.) flanking each end of the cistron to allow for interchangeability of the particular cistron, such as with another cistron or other type of sequence, and upon using standard recombination techniques. In specific embodiments, the restriction enzyme site(s) that flank each of the cistrons is unique to the vector to allow ease of recombination, although in alternative embodiments the restriction enzyme site is not unique to the vector.

In particular embodiments, the vector provides for a unique, second level of modularity by allowing for interchangeability within a particular cistron, including within multiple components of a particular cistron. The multiple components of a particular cistron may be separated by one or more restriction enzyme sites, including those unique to the vector, to allow for interchangeability of one or more components within the cistron. In FIG. 1A, as an example, cistron 2 comprises five separate components, although there may be 2, 3, 4, 5, 6, or more components per cistron. The example in FIG. 1A includes cistron 2 that has five components each separated by unique enzyme restriction sites X₉, X₁₀, X₁₁, X₁₂, X₁₃, and X₁₄, to allow for standard recombination to exchange different components 1, 2, 3, 4, and/or 5. FIG. 1B differs from FIG. 1A by illustrating that there may be multiple restriction enzyme sites between the different components (that are unique, although alternatively one or more are not unique) and that there may be sequence in between the multiple restriction enzyme sites (although alternatively there may not be). In certain embodiments, all components encoded by a cistron are designed for the purpose of being interchangeable. In particular cases, one or more components of a cistron are designed to be interchangeable, whereas one or more other components of the cistron may not be designed to be interchangeable.

In specific embodiments in the examples of FIGS. 1A and 1B, a cistron encodes a CAR molecule or other antigen receptor having multiple components. For example, cistron 2 may be comprised of sequence that encodes a CAR molecule having its separate components represented by component 1, component 2, component 3, etc. The CAR molecule may comprise 2, 3, 4, 5, 6, 7, 8, or more interchangeable components. In a specific example, component 1 in FIG. 1A or 1B encodes a scFv for an antigen of interest; component 2 encodes a hinge; component 3 encodes a transmembrane domain; component 4 encodes a costimulatory domain (although there may also be component 4′ that encodes a second or more costimulatory domain flanked by restriction sites for exchange); and component 5 encodes a signaling domain. In a particular example, component 1 encodes a CD19 scFv; component 2 encodes a IgG1 hinge and/or transmembrane domain; component 3 encodes CD28; and component 4 encodes CD3 zeta.

One of skill in the art recognizes in the design of the vector that the various cistrons and components must be configured such that they are kept in frame when necessary.

In a particular example of FIG. 1A or 1B, cistron 1 encodes a suicide gene; cistron 2 encodes a CAR; cistron 3 encodes a reporter gene; cistron 4 encodes a cytokine; component 1 of cistron 2 encodes a scFv; component 2 of cistron 2 encodes IgG1 hinge; component 3 of cistron 2 encodes CD28; and component 4 encodes CD3 zeta. In alternative embodiments, none of the cistrons encode an antigen receptor.

A restriction enzyme site may be of any kind and may include any number of bases in its recognition site, such as between 4 and 8 bases; the number of bases in the recognition site may be at least 4, 5, 6, 7, 8, or more. The site when cut may produce a blunt cut or sticky ends. The restriction enzyme may be of Type I, Type II, Type III, or Type IV, for example. Restriction enzyme sites may be obtained from available databases, such as Integrated relational Enzyme database (IntEnz) or BRENDA (The Comprehensive Enzyme Information System).

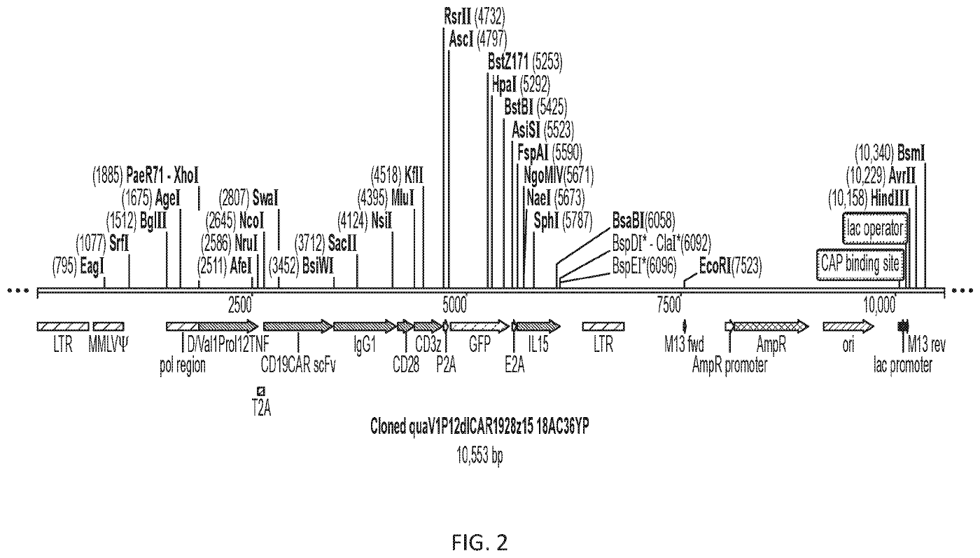

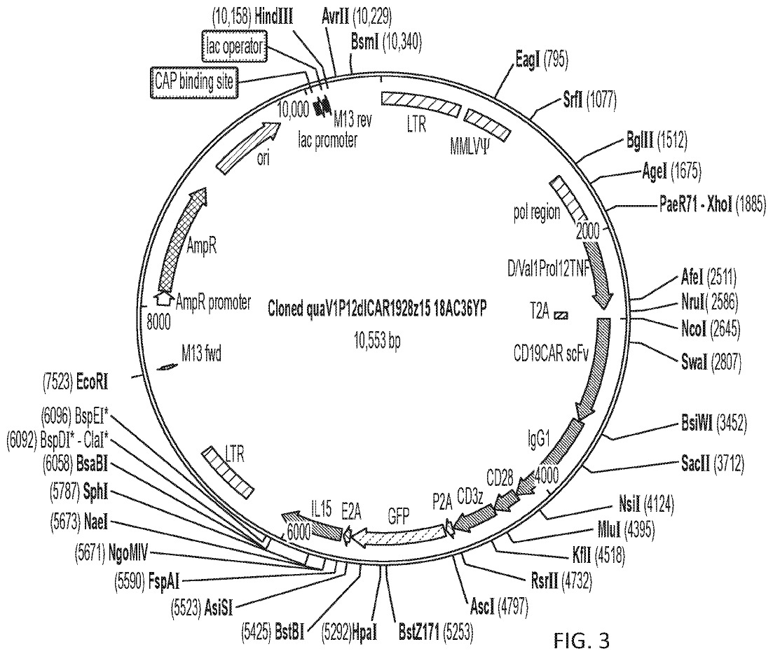

Exemplary vectors are depicted in FIG. 2 and FIG. 3. In FIG. 2, the vector DNA is circular and by convention, position 1 (12 o'clock position at the top of the circle, with the rest of the sequence in clock-wise direction) is set at the start of 5′ LTR.

In embodiments wherein self cleaving 2A peptides are utilized, the 2A peptides may be 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. The mechanism of 2A-mediated “self-cleavage” was discovered to be ribosome skipping the formation of a glycyl-prolyl peptide bond at the C-terminus of the 2A. A highly conserved sequence GDVEXNPGP (SEQ ID NO:5) is shared by different 2As at the C-terminus, and is useful for the creation of steric hindrance and ribosome skipping. Successful skipping and recommencement of translation results in two “cleaved” proteins. Examples of 2A sequences are as follows:

T2A:
(SEQ ID NO: 1)
(GSG)E G R G S L L T C G D V E E N P G P
P2A:
(SEQ ID NO: 2)
(GSG)A T N F S L L K Q A G D V E E N P G P
E2A:
(SEQ ID NO: 3)
(GSG)Q C T N Y A L L K L A G D V E S N P G P
F2A:
(SEQ ID NO: 4)
(GSG)V K Q T L N F D L L K L A G D V E S N P G P

A. Methods of Production

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure.

1. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells may be comprised of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters, for example. In cases wherein the vector is utilized for the generation of cancer therapy, a promoter may be effective under conditions of hypoxia.

a. Promoter/Enhancers

The expression constructs provided herein comprise a promoter to drive expression of the antigen receptor and other cistron gene products. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GenBank®, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

b. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As detailed elsewhere herein, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot-and-mouth disease virus 2A) or a “2A-like” sequence (e.g., Thosea asigna virus 2A; T2A) or porcine teschovirus-1 (P2A). In specific embodiments, in a single vector the multiple 2A sequences are non-identical, although in alternative embodiments the same vector utilizes two or more of the same 2A sequences. Examples of 2A sequences are provided in US 2011/0065779 which is incorporated by reference herein in its entirety.

c. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

d. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

B. Genetically Engineered Antigen Receptors

The vector may encode one or more antigen receptors, such as engineered TCRs, CARs, decoy receptors, cytokine receptors, chimeric cytokine receptors, and so forth. Multiple CARs and/or TCRs, such as to different antigens, may be utilized with the present vector system. A single vector may encode two separate CAR molecules, or a single vector may encode one or more CAR molecules at least one of which has specificity for two non-identical antigens, such as a bispecific CAR, a bispecific TCR, or a bispecific CAR/TCR. The antigen receptors encoded by the vectors of the disclosure, the vectors themselves, and cells harboring the vector are generated by the hand of man and are not present in nature.

In some embodiments, the CAR comprises an extracellular antigen-recognition domain that specifically binds to an antigen. The CAR may be specifically designed to target an antigen of a particular tissue or cell type. In some embodiments, the antigen is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

1. Chimeric Antigen Receptors

In some embodiments, the CAR is encoded by the vector and comprises at least: a) an intracellular signaling domain, b) a transmembrane domain, and c) an extracellular domain comprising at least one antigen binding region.

In some embodiments, the engineered antigen receptors include CARs, including activating or stimulatory CARs, costimulatory CARs (see WO2014/055668), and/or inhibitory CARs (iCARs, see Fedorov et al., 2013). The CARs generally include an extracellular antigen (or ligand) binding domain linked to one or more intracellular signaling components, in some aspects via linker(s) and/or transmembrane domain(s). Such molecules typically mimic or approximate a signal through a natural antigen receptor, a signal through such a receptor in combination with a costimulatory receptor, and/or a signal through a costimulatory receptor alone. The CAR may be first generation, second generation, or third or subsequent generation.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain having one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be derived from human genes used to enhance cellular immunotherapy for human patients. In a specific embodiment, the disclosure includes a full-length CAR cDNA or coding region encoded by the vector. The antigen binding regions or domain may comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion may or may not be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and one or more intracellular signaling domains, such as a CD28 intracellular signaling domain. Other costimulatory receptors include, but are not limited to one or more of CD28, CD27, OX-40 (CD134), DAP10, DAP12, and 4-1BB (CD137). In addition to a primary signal initiated by CD3, an additional signal provided by a human costimulatory receptor inserted in a human CAR may be utilized for full activation of NK cells and could help improve in vivo persistence and the therapeutic success of the adoptive immunotherapy.

In some embodiments, a CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen binding domain or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of tumor associated antigens include CD19, CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, and so forth. In certain embodiments, the CAR may be co-expressed with a cytokine to improve persistence when there is a low amount of tumor-associated antigen. For example, a CAR may be co-expressed with IL-2, IL-21, IL-12, IL-18, or IL-15.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in an expression vector in proper orientation for expression. The polycistronic modular vector of the disclosure may be a viral vector or may not be a viral vector, such as a plasmid. Although for illustrative embodiments the vector detailed herein is a retroviral vector, in other cases the vector is also a viral vector but is instead an adenoviral vector, adeno-associated viral vector, or lentiviral vector, for example.

Any vector can be used to introduce the chimeric construct into immune cells. Suitable vectors for use in accordance with the method of the present disclosure may be non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV. In specific cases, the vector is based on the Moloney Murine Leukemia Virus.

In some aspects, the antigen-specific binding or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as T, NK, iNKT, B, or MSC cells, comprise (i) non-viral gene transfer using an electroporation device (e.g., a nucleofector), (ii) CARs that signal through endodomains (e.g., CD28/CD3-ζ CD137/CD3-ζ or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR⁺ immune cells (Singh et al., 2008; Singh et al., 2011).

2. T Cell Receptor (TCR)

In some embodiments, the genetically engineered antigen receptors include recombinant TCRs and/or TCRs cloned from naturally occurring T cells. A “T cell receptor” or “TCR” refers to a molecule that contains a variable a and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable α chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains comprise a constant domain. For example, like immunoglobulins, the extracellular portion of TCR chains (e.g., α-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or Cₐ, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains may comprise a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3 chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains a and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li et al., 2005). In some embodiments, the TCR or antigen-binding portion thereof can be synthetically generated from knowledge of the sequence of the TCR.

C. Co-Expression of Cytokines

One or more cytokines may be co-expressed from the vector as a separate polypeptide from the antigen receptor. Interleukin-15 (IL-15), for example, is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits AICD.

In some embodiments, the present disclosure concerns co-utilization of CAR and/or TCR vectors with IL-15. In addition to IL-15, other cytokines are envisioned. These include, but are not limited to, cytokines, chemokines, and other molecules that contribute to the activation and proliferation of cells used for human application. NK or T cells expressing IL-15 are capable of continued supportive cytokine signaling, which is critical to their survival post-infusion.

Following genetic modification, the cells may be immediately infused or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the chimeric receptor is expanded ex vivo. The clone selected for expansion demonstrates the capacity to specifically recognize and lyse CD19 expressing target cells. The recombinant immune cells may be expanded by stimulation with IL-2, or other cytokines that bind the common gamma-chain (e.g., IL-7, IL-12, IL-15, IL-21, and others). The recombinant immune cells may be expanded by stimulation with artificial antigen presenting cells. In a further aspect, the genetically modified cells may be cryopreserved.

D. Antigens

Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas as well as autoimmune or alloimmune conditions. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. In some cases, the antigen is associated with an immune-related disorder.

Any suitable antigen may find use in the present method. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include NY-ESO, EGFRvIII, Muc-1, Her2, CA-125, WT-1, Mage-A3, Mage-A4, Mage-A10, TRAIL/DR4, and CEA. In particular aspects, the antigens for the two or more antigen receptors include, but are not limited to, CD19, EBNA, WT1, CD123, NY-ESO, EGFRvIII, MUC1, HER2, CA-125, WT1, Mage-A3, Mage-A4, Mage-A10, TRAIL/DR4, and/or CEA. The sequences for these antigens are known in the art, for example, CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, 3, and MAGE 4 (or other MAGE antigens such as those disclosed in International Patent Publication No. WO99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDCl27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), 1E1 and 1E2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be a bacterial antigen. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Y pestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

E. Suicide Genes

The cells of the present disclosure that have been modified to harbor a vector encompassed by the disclosure may comprise one or more suicide genes. The term “suicide gene” as used herein is defined as a gene which, upon administration of a prodrug or other agent, effects transition of a gene product to a compound which kills its host cell. Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The E. coli purine nucleoside phosphorylase, a so-called suicide gene which converts the prodrug 6-methylpurine deoxyriboside to toxic purine 6-methylpurine, may be used. Other examples of suicide genes used with prodrug therapy are the E. coli cytosine deaminase gene and the HSV thymidine kinase gene.

Exemplary suicide genes include CD20, CD52, EGFRv3, or inducible caspase 9. In one embodiment, a truncated version of EGFR variant III (EGFRv3) may be used as a suicide antigen which can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP). In specific embodiments, a mutant TNF-alpha suicide gene is utilized that encodes for a nonsecretable TNF alpha protein that is expressed on the cell membrane, allowing it to be targeted by an inhibitor, such as an antibody, as described in U.S. Provisional Patent Application 62/769,405, filed Nov. 19, 2018, and in U.S. Provisional Patent Application 62/773,372, filed Nov. 30, 2018, and in U.S. Provisional Patent Application 62/791,464, filed Jan. 11, 2019, all of which are incorporated by reference herein in their entirety.

### III. IMMUNE CELLS

Certain embodiments of the present disclosure concern immune cells that express one or more antigen receptors, such as chimeric antigen receptors (CAR) and/or a T cell receptors (TCR). The immune cells may of any kind, such as T cells (e.g., regulatory T cells, CD4+ T cells, CD8+ T cells, or gamma-delta T cells), NK cells, invariant NK cells, NKT cells, B cells, stem cells (e.g., mesenchymal stem cells (MSCs) or induced pluripotent stem (iPSC) cells). In some embodiments, the cells are monocytes or granulocytes, e.g., myeloid cells, macrophages, neutrophils, dendritic cells, mast cells, eosinophils, and/or basophils. Also provided herein are methods of producing and engineering the immune cells as well as methods of using and administering the cells, such as for adoptive cell therapy, in which case the cells may be autologous or allogeneic with respect to a recipient. Thus, the immune cells may be used as immunotherapy, such as to target cancer cells.

The immune cells may be isolated from subjects, particularly human subjects, including individuals in need of a therapy. The immune cells can be obtained from a subject of interest, such as a subject suspected of having a particular disease or condition, a subject suspected of having a predisposition to a particular disease or condition, or a subject who is undergoing therapy for a particular disease or condition. Immune cells can be collected from any location in which they reside in the subject including, but not limited to, blood, cord blood, spleen, thymus, lymph nodes, and bone marrow. The isolated immune cells may be used directly, or they can be stored for a period of time, such as by freezing.

The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. In particular embodiments, the immune cells are isolated from blood, such as peripheral blood or cord blood. In some aspects, immune cells isolated from cord blood have enhanced immunomodulation capacity, such as measured by CD4-positive or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor, preferably a histocompatibility matched donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from a donor distinct from the subject, the donor is preferably allogeneic, provided the cells obtained are subject-compatible in that they can be introduced into the subject. Allogeneic donor cells may or may not be human-leukocyte-antigen (HLA)-compatible. To be rendered subject-compatible, allogeneic cells can be treated to reduce immunogenicity (Fast et al., 2004).

### IV. METHODS OF TREATMENT

In some embodiments, the present disclosure provides methods for therapy, including immunotherapy, comprising administering an effective amount of immune cells encompassed by the present disclosure that are engineered to express the modular vector system provided herein. In some embodiments, a medical disease or disorder is treated by transfer of an immune cell population that elicits an immune response in a recipient individual. In certain embodiments of the present disclosure, cancer or infection is treated by transfer of an immune cell population that elicits an immune response. Provided herein are methods for treating or delaying progression of cancer in an individual comprising administering to the individual an effective amount of antigen-specific cell therapy (specific to one or more antigens). The present methods may be applied for the treatment of immune disorders, solid cancers, hematologic cancers, and viral infections.

In cases where an individual in need of therapy has cancer, the cancer may be blood cancer or may comprise solid tumors, Tumors for which the present treatment methods are useful include any malignant cell type, such as those found in a solid tumor or a hematological malignancy. Exemplary solid tumors can include, but are not limited to, a tumor of an organ or tissue selected from the group consisting of pancreas, colon, cecum, stomach, brain, head, neck, ovary, kidney, larynx, sarcoma, lung, bladder, melanoma, prostate, skin, thyroid, gall bladder, spleen, liver, bone, endometrium, testes, cervix, esophagus, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of leukemia. Leukemia is a cancer of the blood or bone marrow and is characterized by an abnormal proliferation (production by multiplication) of blood cells, usually white blood cells (leukocytes). It is part of the broad group of diseases called hematological neoplasms. Leukemia is a broad term covering a spectrum of diseases. Leukemia is clinically and pathologically split into its acute and chronic forms.

In certain embodiments of the present disclosure, immune cells are delivered to an individual in need thereof, such as an individual that has cancer or an infection. The cells then enhance the individual's immune system to attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of immune cells can be administered by a number of routes, including parenteral administration, for example, intravenous, intraperitoneal, intramuscular, intrasternal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The immune cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

A. Pharmaceutical Compositions

Also provided herein are pharmaceutical compositions and formulations comprising immune cells (e.g., T cells or NK cells) and a pharmaceutically acceptable carrier.

Pharmaceutical compositions and formulations as described herein can be prepared by mixing the active ingredients (such as the cells) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

B. Combination Therapies

In certain embodiments, the compositions and methods of the present embodiments involve an immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:
A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatrexate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. ARTICLES OF MANUFACTURE OR KITS

An article of manufacture or a kit is provided comprising immune cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### VI. EXAMPLES

The following examples are included to demonstrate particular embodiments of the disclosure. It should be appreciated by those of skill in the art that the techniques disclosed in the examples that follow represent techniques discovered by the inventor to function well in the practice of the methods of the disclosure, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Modular Vector System

A vector was produced as a γ-retroviral transfer vector. The retroviral transfer vector pSFG4 had a backbone based on the pUC19 plasmid (large fragment [2.63 kb] in between HindIII and EcoRI restriction enzyme sites) carrying viral components from Moloney Murine Leukemia Virus (MoMLV) including 5′ LTR, psi packaging sequence, and 3′ LTR. LTRs are long terminal repeats found on either side of a retroviral provirus, and in the case of a transfer vector, brackets the genetic cargo of interest, such as CARs and associated genetic components. The psi packaging sequence, which is a target site for packaging by nucleocapsid, was also incorporated in cis, sandwiched between the 5′ LTR and the CAR coding sequence. Thus, the basic structure of the pSFG4 transfer vector can be outlined as such: pUC19 sequence-5′ LTR-psi packaging sequence-genetic cargo of interest-3′ LTR-pUC19 sequence.

An exemplary vector is depicted in FIGS. 2 and 3. In FIG. 3, the vector DNA is circular and by convention, position 1 (12 o'clock position at the top of the circle, with the rest of the sequence in clock-wise direction) is set at the start of 5′ LTR.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,844,905
U.S. Pat. No. 5,885,796
U.S. Pat. No. 6,207,156
U.S. Pat. No. 6,410,319
U.S. Pat. No. 6,451,995
U.S. Pat. No. 7,070,995
U.S. Pat. No. 7,109,304
U.S. Pat. No. 7,265,209
U.S. Pat. No. 7,354,762
U.S. Pat. No. 7,446,179
U.S. Pat. No. 7,446,190
U.S. Pat. No. 7,446,191
U.S. Pat. No. 8,324,353
U.S. Pat. No. 8,329,867
U.S. Pat. No. 8,354,509
U.S. Pat. No. 8,479,118
U.S. Pat. No. 8,735,553
U.S. Pat. No. 8,008,449
U.S. Pat. No. 8,017,114
U.S. Pat. No. 8,119,129
U.S. Pat. No. 8,252,592
U.S. Pat. No. 8,339,645
U.S. Pat. No. 8,398,282
U.S. Patent Application US2002131960
U.S. Patent Application US20110008369
U.S. Patent Application US2013287748
U.S. Patent Application US20130149337
U.S. Patent Application US20140294898
U.S. Patent Application US2014022021
WO1995001994
WO1998042752
WO199842752
WO2001014424
WO2000037504
WO200014257
WO200037504
WO200114424
WO2012129514
WO2013126726
WO2013166321
WO2013071154
WO2013123061
WO2014055668
WO2014031687
EP Patent Application EP2537416
Ausubel et al., 1996, (eds.) Current Protocols in Molecular Biology, John Wiley & Sons Inc
Camacho et al. (2004) J Clin Oncology 22(145): Abstract No. 2505
Cohen et al. (2005) J Immunol. 175(9): 5799-5808
Chothia et al. (1988) EMBO J. 7(12):3745-55
Davila et al. (2013) Hematol Oncol Clin North Am. 27(2):341-53
Fedorov et al. (2013) Science Translational Medicine, Vol. 5, Issue 215, pp. 215ra172
Hurwitz et al. (1998) Proc Natl Acad Sci USA 95(17): 10067-10071
Jores et al. (1990) Proc Natl Acad Sci USA. 87(23):9138-42
Lefranc et al. (2003) Dev Comp Immunol. 27(1):55-77
Li et al. (2005) Nature Biotechnology volume 23, pages 349-354
Lockey et al. (2008) Front Biosci. 1; 13:5916-27
Mokyr et al. (1998) Cancer Res 58:5301-5304
Parkhurst et al. (2009) Clin Cancer Res. 15(1):169-80
Sadelain et al. (2013) Cancer Discov. 3(4):388-98
Sambrook et al. (2001) Molecular Cloning: A Laboratory Manual, 1st ed., Cold Spring Harbor
Laboratory Press
Singh et al. (2008) Cancer Res.;68(8):2961-71
Singh et al. (2011) Cancer Res. 15; 71(10):3516-27
Turtle et al. (2012) Curr Opin Immunol. 24(5):633-9.
Varela-Rohena et al. (2008) Immunol Res. 42(1-3):166-81.
Wu et al. (2012) Cancer J. 18(2):160-75

What is claimed is:

1. A polycistronic vector comprising at least three cistrons each flanked by one or more restriction enzyme sites, wherein at least one cistron encodes for at least one antigen receptor.

2. The vector of claim 1, wherein two, three, four, or more of the cistrons are translatable into a single polypeptide and the polypeptide is cleaveable into separate polypeptides.

3. The vector of claim 2, wherein four of the cistrons are translatable into a single polypeptide and cleaveable into separate polypeptides.

4. The vector of any one of claims 1-3, wherein adjacent cistrons on the vector are separated by a 2A self cleavage site.

5. The vector of claim 1, wherein each of the cistrons are configured to express separate polypeptides from the vector.

6. The vector of claim 5, wherein adjacent cistrons on the vector are separated by an IRES element.

7. The vector of any one of claims 1-6, wherein at least one of the cistrons on the vector comprises two or more modular components, wherein each of the modular components within a cistron is flanked by one or more restriction enzyme sites.

8. The vector of claim 7, wherein a cistron comprises three, four, or five modular components.

9. The vector of claim 7 or 8, wherein a cistron encodes an antigen receptor having different parts of the receptor encoded by corresponding modular components.

10. The vector of claim 9, wherein a first modular component of a cistron encodes an antigen binding domain of the receptor.

11. The vector of claim 9 or 10, wherein a second modular component of a cistron encodes a hinge region of the receptor.

12. The vector of claim 9, 10, or 11, wherein a third modular component of a cistron encodes a transmembrane domain of the receptor.

13. The vector of any one of claims 9-12, wherein a fourth modular component of a cistron encodes a first costimulatory domain.

14. The vector of any one of claims 9-13, wherein a fifth modular component of a cistron encodes a second costimulatory domain.

15. The vector of any one of claims 9-14, wherein a sixth modular component of a cistron encodes a signaling domain.

16. The vector of any one of claims 1-15, wherein two different cistrons on the vector each encode an antigen receptor.

17. The vector of claim 16, wherein both antigen receptors are encoded by a cistron comprising two or more modular components.

18. The vector of any one of claims 1-17, wherein the antigen receptor is a chimeric antigen receptor (CAR) and/or T cell receptor (TCR).

19. The vector of any one of claims 1-17, wherein the vector is a viral vector or a non-viral vector.

20. The vector of claim 19, wherein the viral vector is a retroviral vector, lentiviral vector, adenoviral vector, or adeno-associated viral vector.

21. The vector of any one of claims 1-20, wherein the vector comprises a Moloney Murine Leukemia Virus (MMLV) 5′ LTR, 3′ LTR, and psi packaging element.

22. The vector of claim 21, wherein the psi packaging is incorporated between the 5′ LTR and the antigen receptor coding sequence.

23. The vector of any one of claims 1-22, wherein the vector comprises pUC19 sequence.

24. The vector of any one of claims 1-23, wherein at least one cistron encodes for a cytokine, chemokine, cytokine receptor, and/or homing receptor.

25. The vector of claim 24, wherein the cytokine is interleukin 15 (IL-15), IL-7, IL-12, IL-21, IL-18, or IL-2.

26. The vector of claim 4, wherein the 2A cleavage site comprises a P2A, T2A, E2A and/or F2A site.

27. The vector of any one of claims 1-26, wherein a cistron comprises a suicide gene.

28. The vector of any one of claims 1-27, wherein a cistron encodes a reporter gene product.

29. The vector of any one of claims 1-28, wherein a first cistron encodes a suicide gene, a second cistron encodes an antigen receptor, a third cistron encodes a reporter gene product, and a fourth cistron encodes a cytokine.

30. The vector of claim 29, wherein different parts of the antigen receptor are encoded by corresponding modular components and wherein a first component of the second cistron encodes an antigen binding domain, a second component encodes a hinge and/or transmembrane domain, a third component encodes a costimulatory domain, and a fourth component encodes a signaling domain.

31. An immune cell that comprises the vector of any one of claims 1-30.

32. The immune cell of claim 31, wherein the immune cell is a T cell, peripheral blood lymphocyte, B cell, NK cell, invariant NK cell, NKT cell, iNKT cell, macrophage, stem cell, or a mixture thereof.

33. The immune cell of claim 32, wherein the stem cell is a mesenchymal stem cell (MSC) or an induced pluripotent stem (iPS) cell.

34. The immune cell of claim 31, wherein the immune cell is derived from an iPS cell.

35. The immune cell of claim 31, wherein the T cell is a CD8+ T cell, CD4+ T cell, or gamma-delta T cell.

36. The immune cell of claim 32 or 35, wherein the T cell is a cytotoxic T lymphocyte (CTL).

37. The immune cell of any one of claims 31-36, wherein the immune cell is allogeneic with respect to an individual.

38. The immune cell of any one of claims 31-36, wherein the immune cell is autologous with respect to an individual.

39. The immune cell of any one of claims 31-38, wherein the immune cell is a human cell.

40. The immune cell of any one of claims 31-39, wherein the immune cell is derived from cord blood, peripheral blood, bone marrow, CD34+ cells, or iPSCs.

41. The immune cell of claim 40, wherein the immune cell is derived from cord blood.

42. The immune cell of any one of claims 31-41, wherein the immune cell is comprised in a population of cells.

43. The immune cell of any one of claims 31-42, wherein the immune cell is comprised in a pharmaceutically acceptable carrier.

44. A method for producing expanding immune cells, comprising:
(a) obtaining a starting population of immune cells;
(b) culturing the starting population of immune cells in the presence of artificial presenting cells (APCs);
(c) introducing a vector of any of claims 1-30 into the immune cells; and
(d) expanding the immune cells in the presence of APCs, thereby obtaining expanded immune cells.

45. The method of claim 44, wherein the starting population of immune cells is obtained by isolating mononuclear cells using a ficoll-paque density gradient.

46. The method of claim 44 or 45, wherein the APCs are gamma-irradiated APCs.

47. The method of claim 44, further comprising cryopreserving a population of the expanded immune cells.

48. A pharmaceutical composition comprising a population of immune cells of any one of claims 31-43 and a pharmaceutically acceptable carrier.

49. A composition comprising an effective amount of immune cells of any one of claims 31-43 for use in the treatment of a disease or disorder in an individual.

50. The composition of claim 49, wherein the disease is cancer or the disorder is an immune-related disorder

51. The use of a composition comprising an effective amount of immune cells of any one of claims 31-43 for the treatment of cancer or an immune-related disorder in an individual.

52. A method of treating a disease or disorder in an individual, comprising administering an effective amount of immune cells of any one of claims 31-43 to the individual.

53. The method of claim 52, wherein the disease or disorder is a cancer, autoimmune disorder, graft versus host disease, allograft rejection, or inflammatory condition.

54. The method of claim 53, wherein the autoimmune disorder is an inflammatory condition and the immune cells have essentially no expression of glucocorticoid receptor.

55. The method of claim 54, wherein the subject has been or is being administered a steroid therapy.

56. The method of any one of claims 52-55, wherein the immune cells are autologous with respect to the individual.

57. The method of any one of claims 52-55, wherein the immune cells are allogeneic with respect to the individual.

58. The method of any one of claim 52, 53, 56, or 57, wherein the disease is a cancer.

59. The method of claim 58, wherein the cancer is a solid cancer or a hematologic malignancy.

60. The method of claim 59, further comprising administering at least a second therapeutic agent to the individual.

61. The method of claim 60, wherein the second therapeutic agent comprises chemotherapy, immunotherapy, surgery, radiotherapy, or biotherapy.

62. The method of any one of claims 52-61, wherein the immune cells are administered to the individual intravenously, intraperitoneally, intratracheally, intratumorally, intramuscularly, endoscopically, intralesionally, percutaneously, subcutaneously, regionally, by perfusion, or by direct injection.

63. The method of claim 61 or 62, wherein the second therapeutic agent is administered to the individual intravenously, intraperitoneally, intratracheally, intratumorally, intramuscularly, endoscopically, intralesionally, percutaneously, subcutaneously, regionally, by perfusion, or by direct injection.

64. A polycistronic vector comprising at least four cistrons each flanked by one or more restriction enzyme sites, wherein at least one of the cistrons on the vector comprises two or more modular components, wherein each of the modular components within a cistron is flanked by one or more restriction enzyme sites.
